(19) United States
(12) Patent Application Publication
(10) Pub. No.: US 2007/0048364 A1
(43) Pub. Date: Mar. 1, 2007

(54) Free flowing granules containing carbomer

(76) Inventors: Yingxu Peng, Memphis, TN (US); Yichun Sun, Germantown, TN (US); James R. Johnson, Germantown, TN (US); Atul J. Shukla, Cordova, TN (US)

Correspondence Address: HOWARD EISENBERG, ESQ., 2206 APPLEWOOD COURT, PERKASIE, PA, 18944, US

(21) Appl. No.: 11/197,102

(22) Filed: Aug. 4, 2005

(51) Int. Cl. A61K 9/48; A61K 9/20; A61K 9/14

(52) U.S. Cl. 424/451; 424/464; 424/489; 264/109

(57) ABSTRACT

Methods for making granules containing a carbomer whereby the carbomer is mixed with a hot-melt binder that does not lower the glass transition temperature of the carbomer more than 20° C., the binder is subjected to a temperature at which the hot-melt binder melts or becomes tacky, thus binding the carbomer to form an agglomerated powder, and permitting granules to form from the agglomerated powder. This subjecting of the binder to the temperature may be after the binder is mixed with the carbomer or the carbomer may be mixed with the binder at a time when the binder is at a temperature at which it will melt or become tacky. The granules may contain a biologically active substance either intragranularly or extragranularly and may be used to make personal care products or pharmaceutical dosage forms, such as controlled release dosage forms which may have bioadhesive properties.

### FIELD OF THE INVENTION

The invention pertains to the field of granulation processes to produce free flowing granules. In particular, the invention pertains to the field of production of granules containing carbomers.

### BACKGROUND OF THE INVENTION

The process of manufacturing pharmaceutical products such as tablets and capsules often requires improving flow and/or compressibility of a powder mixture. Tabletting involves compression of blends of powdered materials provided the blends have adequate flow characteristics and compactibility. Powdered materials that have adequate flow characteristics and compactibility may be processed into tablets by a direct compression method. However, if either or both traits, adequate flow and/or adequate compactibility, are missing from the powder blend, then a suitable granulating technique is employed to granulate the powder blend prior to compression so that the resulting granules have improved flow and compactibility.

Carboxyvinyl polymers (CLPs) are a family of water-swellable, high molecular weight, crosslinked homopolymer and copolymer resins based on an acrylic acid backbone. The characteristics of these resins may be modified by modifying the crosslinker types and level, as well as the amounts and characteristics of the hydrophobic co-monomers. Carbomer is a generic name that is often used to describe such polymers. Carbomers may be divided into the following categories: (a) homopolymers of acrylic acid crosslinked with allyl sucrose or allyl pentaerythritol (Carbopol®); b) polycarbophils (calcium salt and acid form) of acrylic acid crosslinked with divinyl glycol (Noveon®); c) copolymers of acrylic acid with minor levels of long chain alkyl acrylate crosslinked with allyl pentaerythritol (Carbopol® copolymers and Pemulen® polymeric emulsifiers). The polymers also have a number of synonyms, such as Acritamer®, acrylic acid polymer, Carbopol®, carboxy polymethylene, polyacrylic acid, carboxyvinyl polymer, Pemulen®, and Ultrez®. A number of agencies, including the USP-NF, and United States Adopted Names Council (USAN) have adopted the generic name “carbomer” for crosslinked homopolymers and copolymers based on acrylic acid backbone. The terms carbomer and CLP are used herein synonymously.

CLPs have been used worldwide for many years in a diverse range of pharmaceutical applications, personal care and home care products. CLPs are used as thickening agents. At very low concentrations (less than 1%), the CLP produces a very wide range of viscosities and flow properties of topical lotions, creams and gels, oral suspensions. They are also used as thickening agents in transdermal gel reservoirs. CLPs are used as suspending agents for insoluble ingredients in oral suspensions and topical preparations. CLPs are also used to stabilize emulsions by acting as both emulsifying and thickening agents. Also, because of their strong adhesive characteristics, CLPs are used as bioadhesives for buccal, ophthalmic, intestinal, nasal, vaginal and rectal applications. Additionally, CLSs are used as controlled release agents to control the release of biologically active substances (BAS) from tablets and capsules.

Thus, the use of CLPs in pharmaceutical products provides several advantages. They provide controlled drug release from granules, tablets, or capsules. They also provide flexibility to add additional ingredients to a pharmaceutical formulation without dramatically increasing the size of the final dosage form, since only a low concentration of such polymer is needed compared to the other hydrophilic polymers, to achieve controlled release. In fact, the use of CLPs at low concentrations can reduce the size of a final dosage form such as tablets or capsules, thus minimizing packaging and shipping costs, which ultimately results in lower overall cost of the final dosage form. A reduced size of a final dosage form such as a tablet or capsule can also make it easier to swallow for geriatric or pediatric patients.

Presently marketed CLPs are flocculated powders with an average particle size of about 0.2 μm in diameter. These flocculated powders have poor flow because they are cohesive and possess static charges. Also, because of the inherently crosslinked structure of the polymers, the CLP particles clump very easily when mixed with water or other polar solvents. This is because the outside of the clumps becomes hydrated or wetted, whereas the interior of the clumps remains as a dry core. In addition, when wetted, the hydrated outer layer is viscous and prevents water or other polar solvents from wetting the interior of the clumps. Thus, carbomer powders are difficult to dissolve or disperse in hydrophilic solvents. The CLP powder is also light and fluffy, and hence difficult to handle. Therefore, it is desirable to provide a granulated, densified CLP in order to improve the flow characteristics as well as ameliorate the aforementioned problems. The handling characteristics of a CLP-containing material will also be dramatically improved by granulating and densifying the presently available CLP. Moreover, a granulated CLP with BAS can be used as a drug delivery system as well. However, the aforementioned undesirable physical properties of CLP make it difficult to granulate these polymers using traditional granulation techniques.

The three most commonly used methods of granulation include wet granulation, dry granulation, and hot-melt granulation.

In wet granulation, a powder mixture is moistened with water, an organic solvent, or an aqueous/organic binder solution which results in a wet mass from which the solvent is then evaporated, typically by drying in an oven, microwave, or an infrared or fluid-bed dryer. The resulting granules can then be compressed into tablets or filled into capsules with or without additional excipients. However, due to the problems of clumping when CLPs are exposed to water, it is not feasible to granulate these polymers with water or aqueous binder solutions using conventional wet granulation techniques, especially when the concentration of CLP in the formulations is high.

Dry granulation is achieved typically by either a slugging or a roller compaction process. With slugging, a powder mixture is compressed into slugs, large tablets about 1 inch in diameter. The tablets are then broken and sieved through appropriate sieves to obtain granules of the desired size. The sieved granules are then compressed into tablets or filled into capsules with or without additional excipients.

With roller compaction, a powder is passed through rollers to form a compacted sheet of the material. The compacted sheet is then passed through a comminuting mill fitted with an appropriate size of sieve in order to obtain granules of the desired size. The resulting granules are then compressed into tablets or filled into capsules with or without additional excipients.

Dry granulation methods have several disadvantages. They require additional equipment. Moreover, with these methods it is often difficult to control the size of the resultant granules and loss of starting material is usually greater with dry granulation than with other methods. The dry granulation process also produces significant amounts of dust, which represents loss of materials and may cause a hazard to equipment and personnel. One grade of granulated carbomer produced by the roller compaction technique has been marketed by Noveon, Inc. (Cleveland, Ohio).

Hot-melt granulation utilizes a material referred to as a hot-melt binder, which is a solid or semi-solid at room temperature and which melts at a temperature below that at which the BAS of interest melts. Typically, the binder melts at a temperature between 30° C. and 200° C. A solvent such as water or an organic compound is not necessary to initiate binding in this method.

The hot-melting binder, when heated to a sufficiently high temperature, liquifies or becomes tacky. This tacky and/or liquified binder spreads itself over the surface of the powdered or particulate matter in a mixture and forms agglomerates of the mixture, which upon cooling, forms a solid granulated mass in which the powder or particulate starting materials are bound. The resultant granules can then be provided to a tablet press, mold, or encapsulator, such as a capsule filling machine, for preparing the desired dosage form with or without additional excipients.

Hot-melt granulation utilizes commonly used granulating equipment, such as low shear mixer, high shear mixer granulator, fluidized bed granulator, rotating pan, or extruder. The energy to melt the binder may come from heat dissipated from circulating hot liquid, such as water or oil, steam, hot air, or heat generated from friction during the granulation process.

Hot-melt techniques eliminate the disadvantages present with wet and dry granulation techniques. Additional solvents and extensive drying times associated with wet granulation methods are eliminated as are the dust and loss problems associated with dry granulation methods. Moreover, hot-melt techniques permit the production of denser granules in a shorter time period than is possible with other granulation methods.

Because of its advantages, hot-melt granulation techniques have been extensively utilized and several adaptations of this technique have been made. Several patents disclose the use of hot-melt and similar granulation techniques to produce or to modify granules for immediate release and delayed release pharmaceutical compositions. The patents include Speiser, U.S. Pat. No. 4,013,784; Blichare, U.S. Pat. No. 4,132,753; Ahrens, U.S. Pat. No. 4,935,246; Kristensen, U.S. Pat. No. 5,476,667; Humer, U.S. Pat. No. 5,667,807; Royce, U.S. Pat. No. 5,403,593; and Heafield, U.S. Pat. No. 6,143,328, each of which is incorporated herein by reference.

Blichare discloses contacting a wax-like material with a powdered medicament at a temperature above the melting point of the wax-like material. This results in the powdered medicament sinking into the molten surface of the wax-like pieces to form spherical granules having an interior of a medicament surrounded by a coating of the wax-like material. Such granules contain up to about 80% active therapeutic ingredient and are suitable for time-release dosage forms.

Kristensen discloses a two-step process by which granules containing high concentrations of BAS may be obtained. A BAS in a cohesive form, such as having a mean particle size less than 30 microns, is mixed with a binder. The mixture is heated to melt the binder and form overwetted spherical pellets. Additional quantities of BAS are then added to the overwetted spherical pellets to obtain the desired pellets, which may have a ratio of BAS:binder as high as 95:5.

Hurner discloses a hot-melt granulation method whereby an active compound having a melting point between 30° C. and 200° C. fulfils the function of a binder. According to the method of Hurner, a low-melting active compound and inactive compounds such as binders, fillers, and disintegrants, are mixed and heated to a temperature at which a part of the active compound itself is melted. Granules are formed by extrusion, a process which requires over-wetting. Thus, the low-melting active compound acts as the binder according to the invention of Hurner.

Royce discloses a hot-melt granulation technique utilizing 5 to 90% concentration of a hydrophilic cellulose ether polymer, 5 to 50% of a granulating medium (binder), and a therapeutically active medicament. A mixture containing these components, plus additional excipients, is heated for a time sufficient to completely liquefy the mixture, which is then cooled to room temperature and formed into granules.

Several patents disclose processes whereby granules containing a carbomer may be produced. Adams, U.S. Pat. No. 6,492,488, discloses a dry granulation process for making control release granules containing a carbomer. Busson, U.S. Pat. No. 6,534,087, discloses methods for the production of pharmaceutical compositions in which a solution or homogenous dispersion is prepared, the solution or dispersion is exposed to changes in pressure so as to expand the solution or dispersion, and the expanded solution or dispersion is then stabilized. Busson discloses that the solution or dispersion may contain a carbomer and that the compositions may be processed into granules. Barry et al., U.S. Pat. No. 5,051,263, discloses a wet granulation process for making a granule whereby a pharmacologically active substance is mixed with a carbomer to produce a cohesive product which is then extruded, chopped into lengths, spheronized, and dried to produce control release granules. Salecki-Gerhardt et al., U.S. Pat. No. 5,919,489, discloses a wet granulation process for making a granule whereby an antibiotic is mixed with a carbomer, which resultant mixture is wetted and blended to form granules which are then dried.

Akiyama et al., U.S. Pat. No. 6,428,813, discloses the production of granules containing (a) a polyglycerol fatty acid ester or a lipid, (b) a viscogenic agent which may be a carbomer, (c) a curdlan or a low-substituted hydroxypropylcellulose, and (d) an active ingredient. The granules may be made by producing a composition containing these four components by melting the polyglycerol fatty ester or lipid and then adding the other three components, either together or serially, to form a dispersion. Granules may be formed from the dispersion in a granulating machine or by spray chilling, a process by which the dispersion is dripped onto a revolving rotary disk.

The composition of Akiyama may also be made by a process that utilizes melt granulation technology. According to this process, the polyglycerol fatty acid ester or lipid is heated to a temperature near its melting point and is then granulated, such as by spray chilling. The granules produced are then suspended with the other three components, the viscogenic agent, the curdlan or HPC, and the active ingredient, to provide an adherent matrix-drug system in which the three components form a coating on the polyglycerol fatty acid ester or lipid granules. Akiyama does not disclose the formation of carbomer-containing granules by melt granulation.

The prior art does not disclose a melt granulation process whereby blank granules, that is those granules lacking a biologically active substance, containing carbomer are produced. Such blank granules are easy to handle and useful to increase the viscosity of personal care and household products such as shampoos, soaps, or hand lotions. Additionally, such blank granules are useful in that a BAS may be added extragranularly for the production of tablets and capsules having a controlled release property. Blank granules may also be used to make shampoos or other personal care products.

### BRIEF DESCRIPTION OF THE FIGURES

FIG. 1 is a graph showing the effect of polymer concentration on the dissolution profile of propranolol hydrochloride from carbopol matrix tablets in Simulated Intestinal Fluid (SIF) using basket method at 100 rpm (37° C.).

FIG. 2 is a graph showing the effect of polymer concentration on the dissolution profile of propranolol hydrochloride from carbopol matrix tablets in Simulated Gastric Fluid (SGF) using basket method at 100 rpm (37° C.).

FIG. 3 is a graph showing the effect of polymer concentration on the dissolution profile of propranolol hydrochloride from carbopol matrix tablets in SIF using basket method at 100 rpm (37° C.).

FIG. 4 is a graph showing the effect of polymer concentration on the dissolution profile of propranolol hydrochloride from carbopol matrix tablets in SGF using basket method at 100 rpm (37° C.).

FIG. 5 is a graph showing the effect of different types of binders on dissolution profile of propranolol hydrochloride from carbopol matrix tablets with intra-granular drug and filler.

FIG. 6 is a graph showing the effect of different types of binders on dissolution profile of propranolol hydrochloride from carbopol matrix tablets with extra-granular drug and filler.

FIG. 7 is a graph showing the effect of binder concentration on the dissolution profile of propranolol hydrochloride from carbopol matrix tablets.

FIG. 8 is a graph showing the effect of different types of fillers on dissolution profile of propranolol hydrochloride from carbopol matrix tablets with extra-granular drug and filler.

### DESCRIPTION OF THE INVENTION

In one embodiment, the invention is a hot-melt granulation process for making granules containing a carbomer. According to this embodiment, a carbomer is mixed with a hot-melt binder, which binder is a solid or semi-solid at room temperature, and the binder is subjected to a temperature at which the hot-melt binder melts or becomes tacky, thus binding the carbomer to form an agglomerated powder, and permitting granules to form from the agglomerated powder. This subjecting of the binder to the temperature may be after the binder is mixed with the carbomer or the carbomer may be mixed with the binder at a time when the binder is at a temperature at which it will melt or become tacky. Preferably, the concentration of the hot-melt binder in the mixture is at or above that which will agglomerate the powder but below that which will result in over-wetting of the mixture upon the melting of the binder.

According to this embodiment, the method of the invention produces blank carbomer-containing granules, that is the granules contain no, or essentially no, biologically active substance (BAS). The blank granules produced in accordance with the method of the invention are useful in many applications. For example, they may be used to increase the viscosity of personal care and household products such as shampoos, soaps, or hand lotions. Additionally, such blank granules are useful in that a BAS may be added extragranularly for the production of tablets or capsules having a controlled release property.

The blank granules of the invention may also be used as a base to which a formulator may add other ingredients, such as colors, flavors, adsorbents, absorbents, or other solid or semi-solid ingredients including BAS for shampoos, foods, cosmetics, pharmaceuticals, and other products. Thus, the blank granules of the invention containing such other ingredients may then be combined with water and/or one or more other appropriate solvents with or without additional extragranular ingredients to obtain a product.

In accordance with this embodiment of the invention, a carbomer is mixed with a hot-melt binder, which binder is a solid or semi-solid at room temperature, and the binder is subjected to a temperature at which the hot-melt binder melts or becomes tacky. The subjecting of the binder to a temperature at which the binder melts or becomes tacky may be after mixing with the carbomer or the carbomer may be mixed with the binder at a time when the binder is at a temperature at which it will melt or become tacky. If desired, the mixture may contain additional excipients, such as fillers, disintegrants, coloring agents, glidants, adsorbents, flavoring agents, controlled release agents such as polymers, waxes, and gums, or additional dry binders. The heat supplied to the mixture may be from any source, such as from an external source like circulating hot liquid (such as water or oil) through a jacketed bowl, hot air or steam, or microwave, infrared sources, heating tape, tumbling or energy released from high-shear mixing due to friction. Following or during the process of agglomeration, granules are formed, which are cooled to ambient temperature. The concentration of the hot-melt binder in the mixture is at or above that which will agglomerate the powder and is preferably, but not necessarily, below that at which will result in over-wetting of the mixture upon the melting of the binder.

The concentration of carbomer in the powder mixture may be varied depending upon the desired use of the granules. Thus, the concentration of carbomer in the mixture on a w/w basis may be as low as 0.1% or even lower if desired or as high as 20% to 50% or higher, such as up to 90%. Preferably, the concentration of carbomer may be between 5% and 30%. The concentration of hot-melt binder may vary depending on several variables such as the type of the particular binder used and its properties, like the melting point and hydrophobicity/hydrophilicity of the binder, as well as the presence or absence of additional excipients or additives and the physical properties of the excipients or additives in the powder mixture.

As defined herein, a “suitable hot-melt binder” for the invention is one that, when mixed with a carbomer, does not result in a lowering of the glass transition temperature (Tg) of the carbomer of more than 20° C. Preferably, the hot-melt binder does not lower the Tg of the carbomer more than 15° C. Most preferably, the hot-melt binder, when mixed with a carbomer, does not lower the Tg of the carbomer at all. Hot-melt binders that raise the Tg of the carbomer are also suitable for the invention. When a suitable hot-melt binder is mixed with a carbomer, discrete free flowing granules are produced. As shown in Table 1, such binders, upon mixing with a carbomer also typically result in an increase in bulk density of the resulting agglomerated granular product.

A preferred group of suitable binders includes waxes. Such waxes may be derived from animal or plant sources, hydrogenated oils, natural, semi-synthetic or synthetic waxes, carnauba wax, bees, paraffin, stearic acid and salts thereof, cetyl alcohol, saturated polyglycolized glycerides, semi-synthetic glycerides, glyceryl esters of fatty acids, glyceryl behenate, glyceryl di and tri stearate, glyceryl palmitostearate, lauroyl macrogol-32 glycerides, stearoyl macrogol-32 glycerides, polyethylene glycol esters of fatty acids such as PEG-32 glyceryl laurate, PEC-32 glyceryl palmitostearate, PEG-32 glyceryl stearate, cetyl palmitate, stearyl alcohol, and solid or semisolid surfactants.

When carbomers are combined with binders that are not suitable, as defined above, suitable granules are not obtained. On the other hand, it is conceived that binder materials comprising a binder material that, by itself, lowers the glass transition temperature of carbomer more than 20° C. may be used in combination with one or more other additives, which may or may not be in itself a binder. Such combinations are suitable as binders in accordance with the invention, so long as the combination of binders with additives, when used together, does not result in a decrease of the Tg of carbomer more than 20° C., preferably not more than 15° C., and most preferably results in no decrease of the Tg of carbomer. Such combinations will result in discrete free flowing granules. Typically the bulk density of the resulting agglomerated granular product will increase.

Table 1 shows the characteristics of various binders that, by themselves, are suitable or not suitable as the hot-melt binder in accordance with the invention.

TABLE 1

| | | | Powder | After | % | | | |
|---|---|---|---|---|---|---|---|---|
| | | | Mixture | Granulation | Change | | | |
| | | Carbopol | Bulk Density | Bulk Density | in bulk | | Visual | |
| Binder | % | 971P (%) | (g/mL) | (g/mL) | density | Tg (° C.) | Observation | Comments |
| NONE | 0 | 100% | 0.22 | 0.22 | 0 | 115 | Cohesive fluffy | Raw Material |
| | | | | | | | powder with poor | |
| | | | | | | | flow | |
| Polawax A31 | 40 | 60 | 0.22 | 0.52 | 140.1 | 137.1 | Discrete free- | Suitable Binder |
| | | | | | | | flowing granules | |
| Polawax NF | 40 | 60 | 0.21 | 0.47 | 128.6 | 134.0 | Discrete free- | Suitable Binder |
| | | | | | | | flowing granules | |
| Syncrowax HR-C | 40 | 60 | 0.27 | 0.54 | 102.8 | 157.0 | Discrete free- | Suitable Binder |
| | | | | | | | flowing granules | |
| Crodamol SS | 40 | 60 | 0.23 | 0.46 | 99.0 | 151.9 | Discrete free- | Suitable Binder |
| | | | | | | | flowing granules | |
| IMWITOR 900 | 45 | 55 | 0.28 | 0.54 | 90.7 | 141.3 | Discrete free- | Suitable Binder |
| | | | | | | | flowing granules | |
| Sterotex K | 40 | 60 | 0.29 | 0.53 | 85.4 | 133.3 | Discrete free- | Suitable Binder |
| | | | | | | | flowing granules | |
| SEFA | 50 | 50 | 0.30 | 0.54 | 82.1 | 124.8 | Discrete free- | Suitable Binder |
| | | | | | | | flowing granules | |
| PEG-20-Stearate | 40 | 60 | 0.24 | 0.43 | 79.6 | 99.8 | Discrete | Acceptable binder |
| | | | | | | | elastic granules | |
| PEG-150- | 40 | 60 | 0.22 | 0.25 | 12.2 | 87.6 | Rubbery Mass | Unsuitable Binder |
| Di-Stearate | | | | | | | | |
| Pluronic F68 | 40 | 60 | 0.21 | 0.24 | 12.1 | 94.6 | Rubbery Mass | Unsuitable Binder |
| Crodesta F-160 | 40 | 60 | 0.24 | 0.24 | −3.3 | 90.4 | Rubbery Mass | Unsuitable Binder |
| Na2HPO4-7H2O | 40 | 60 | 0.20 | 0.18 | −9.9 | 84.7 | Rubbery Mass | Unsuitable Binder |
| PEG 8000 | 40 | 60 | 0.21 | 0.48 | 128.6 | 89.8 | Sticky Rubbery | Unsuitable Binder |
| | | | | | | | Mass | |

Table 2 shows the characteristics of suitable and unsuitable binders based on the changes in Tg, bulk density and flow characteristics of the resulting product

TABLE 2

| | BULK DENSITY | FLOW OF | |
|---|---|---|---|
| Tg OF | OF THE | THE | SUITABILITY |
| CARBOMER | RESULTING | RESULTING | OF THE |
| UPON MIXING | PRODUCT | PRODUCT | BINDER |
| Increase | Increase | Free flowing | Suitable |
| | | discrete | |
| | | granules | |
| Remains the | Increase | Free flowing | Suitable |
| same | | discrete | |
| | | granules | |
| Decrease less | Increase | Free flowing | Suitable |
| than 20° C. | | discrete | |
| | | granules | |
| Decrease more | Marginally | Rubbery mass | Unsuitable |
| than 20° C. | increase or | | |
| | remains | | |
| | the same | | |
| Decrease more | Increase | Rubbery mass | Unsuitable |
| than 20° C. | | | |

The concentration of the hot-melt binder in the mixture, both in absolute terms and in relation to the concentration of carbomer, may be varied depending on several factors, including the intended use of the granules, the properties of the hot-melt binder, such as melting point and hydrophobicity/hydrophilicity, and the presence or absence of additional excipients or additives.

If additional excipients are desired, such as (a) fillers like lactose, microcrystalline cellulose, starch or calcium phosphate salts, (b) disintegrants like cross-linked carboxymethylcellulose such as sold under the brand name AC-DI-SOL® (FMC Corporation, Philadelphia, Pa.), sodium starch glycolate such as sold under the brand name ExploTab® (J. Rettenmaier USA, Schoolcraft, Mich.), or cross-linked polyvinylpyrrolidone such as crospovidone (BASF Corporation) or Polyplasdone® (International Specialty Products), (c) glidants such as silicon dioxide or talc, (d) flavoring agents, (e) coloring agents, (f) other controlled release agents such as polymers, waxes, and gums, (g) dry binders such as polyvinylpyrrolidone, hydroxypropylmethylcellulose, hydroxypropylcellulose, hydroxyethylcellulose, sodium carboxymethylcellulose (Na—CMC), methylcellulose, microcrystalline cellulose, ethylcellulose, or waxy material such as carnauba wax, or stearyl alcohol, or various combinations of the dry binders or one or more of the hydrophilic cellulose ether polymers disclosed in Royce, U.S. Pat. No. 5,403,593 (incorporated herein by reference), (h) lubricants such as stearic acid, magnesium or calcium stearate, or hydrogenated vegetable oils, or (i) adsorbants, these additional excipients may be added extra-granularly or intra-granularly.

In another embodiment, the invention is a carbomer-containing granule, which is substantially free of a biologically active substance. Preferably, such granules, referred to as blank granules, are made by the above-described method of the invention. The blank granules are free flowing and may be used as a component in a variety of personal care and household products, such as shampoos, soaps, and lotions. The blank granules may be used as suspending agents, emulsifying agents, or rheology modification agents to thicken or stabilize liquid or semisolid dosage forms such as emulsions, lotions, creams, ointments, gels and ophthalmic drops. Additionally, the blank granules may be combined with a biologically active substance, which is added extragranularly before converting it into a dosage from such as tablet, capsule, suppository or even a solution, to provide the resulting dosage forms with a controlled release property. For example, the granules may be used as the controlled release agents in solid dosage formulations, such as bio-adhesive delivery systems for buccal, nasal, vaginal, rectal, internal, or ophthalmic applications.

In another embodiment, the invention is a method for producing a controlled release BAS-containing tablet or capsule. According to this embodiment, one or more BAS is added extragranularly to blank carbomer-containing granules, which granules are produced by the above-described method. According to this method, the concentration of carbomer in the granules is sufficient to provide controlled-release properties to the BAS in the tablet or capsule, such as when the tablet or capsule is swallowed or otherwise exposed to a liquid such as water or an organic solvent. Typically, but not necessarily, the concentration of carbomer in the granules prior to addition of the BAS is about 10% or more.

In another embodiment, the invention is a method for making granules containing one or more carbomers and a BAS by a hot-melt granulation process. According to this embodiment, one or more carbomers, one or more hot-melt binders that are suitable for the invention as described above, and one or more BAS are combined to form a mixture. The binder is subjected to a temperature at which the binder melts or becomes tacky, thus binding the carbomer and the BAS to form granules. The subjecting of the binder to a temperature at which the binder melts or becomes tacky may be after mixing with the carbomer or the carbomer may be mixed with the binder at a time when the binder is at a temperature at which it will melt or become tacky. Preferably, the concentration of the binder in the mixture is at or above that which will agglomerate the powder but below that at which will result in over-wetting of the mixture upon the melting of the binder.

As described above for the method for producing blank granules, if desired, the powder mixture may contain additional excipients, such as fillers, disintegrants, controlled release agents such as polymers, waxes, and gums, coloring agents, glidants, flavoring agents, or dry binders and lubricant. These additional excipients may be added either before or after granulation, but before compression into tablets. The heat supplied to the mixture may be from any source, such as from an external source like circulating hot liquid (such as water or oil), hot air or steam, or microwave, infrared sources, or heating tape, tumbling, or heat generated from friction during the granulation process. Following or during the process of agglomeration, granules are formed, which are cooled to ambient temperature. The concentration of the binder in the mixture is at or above that which will agglomerate the powder and is preferably, but not necessarily, below that at which will result in over-wetting of the mixture upon the melting of the binder.

In another embodiment, the invention is a carbomer-containing granule which granule further contains one or more biologically active substance (BAS). Preferably, such granules are made by the above-described method of the invention and are free flowing. The granules may be further processed into pharmaceutical dosage forms such as capsules, tablets, suppositories, pessaries, or gels.

In another embodiment, the invention is a hot-melt granulation method for producing a controlled release granule or a pharmaceutical dosage form, with or without bioadhesive properties, such as a tablet or capsule. According to this embodiment, a binder that is suitable for the invention as described above and one or more carbomers, as disclosed above, are combined to form a mixture, the binder is subjected to a temperature at which the binder melts or becomes tacky, thus agglomerating the mixture, and granules are formed from the heated mixture. The subjecting of the binder to a temperature at which the binder melts or becomes tacky may be after mixing with the carbomer or the carbomer may be mixed with the binder at a time when the binder is at a temperature at which it will melt or become tacky. Other controlled release agents, including polymers, waxes, gums, and buffer salts which modulate the controlled release properties of the carbomer, in addition to the hot-melt binder, may be included in the mixture before the mixture is heated and granulated. Alternatively, the polymer, gum, or wax may be added extragranularly. If desired, a BAS may either be added to the mixture so that the BAS forms a part of the granule or may be added extragranularly. Additional excipients, as described above, may be combined with the granules intragranularly or extragranularly, if desired. Materials that are suitable for this embodiment of the invention include polymers, waxes, gums, and buffer salts. The inclusion of such controlled release agent modulates the control release nature of a granule or other pharmaceutical dosage form for oral administration. Examples of suitable controlled release agent for this embodiment of the invention include polyvinyl pyrrolidone, polyvinyl alcohol, ethylene vinyl acetate copolymer, cellulose derivates, hydroxyethylcellulose, hydroxypropylcellulose, hydroxypropylmethylcellulose, methylcellulose, sodium carboxymethylcellulose, cellulose esters, cellulose acetate, cellulose proprionate, cellulose acetate butyrate, and polymethyacrylates.

In another embodiment, the invention is a granule containing a carbomer, which granule includes a material that modulates the controlled-release nature of a carbomer-containing granule, which material may be, for example, a polymer, a wax, a gum, or a buffer salt. Preferably, the granule is made by the aforementioned method of the invention. Alternatively, the polymer, gum, or wax may be added extragranularly. If desired, a BAS may either be added to the mixture so that the BAS forms a part of the granule or may be added extragranularly. The aforementioned additional excipients, may be combined with the granules intragranularly or extragranularly, if desired.

Preferably, the granule containing carbomer includes one or more materials that modulate the controlled-release nature of carbomer-containing granules at a low pH environment (pH<6.0). The material may be, for example, a polymer, a wax, a gum, or a salt. Preferably, the granule is made by the method of the invention described above. Alternatively, the polymer, gum, or wax may be added extragranularly. If desired, a BAS may either be added to the mixture so that the BAS forms a part of the granule or may be added extragranularly. Additional excipients, as described above, may be combined with the granules intragranularly or extragranularly, if desired. Examples of suitable materials for this embodiment of the invention include polyvinyl pyrrolidone, polyvinyl alcohol, ethylene vinyl acetate copolymer, cellulose derivates, hydroxyethylcellulose, hydroxypropylcellulose, hydroxypropylmethylcellulose, methylcellulose, sodium carboxymethylcellulose, cellulose esters, cellulose acetate, cellulose proprionate, and cellulose acetate butyrate, polymethyacrylates, enteric coating materials, or salts.

If desired, the methods and granules of the several embodiments of the invention may be free of curdlan and low-substituted hydroxypropylcellulose, such as described in Akiyama, U.S. Pat. No. 6,428,813. Alternatively, the methods and granules may include such curdlan or low-substituted hydroxypropylcellulose.

Examples of BAS that are suitable for the various embodiments of the invention include, but are not limited to steroids, hormones, antipsychotic agents, agents that act on the central nervous system (CNS—agents), narcotic agonists and antagonists, psychotherapeutic agents, fertility regulating agents, antibodies and antigens, anesthetics, analgesics and antipyretics, anticonvulsants, antiinfective and antibiotics, antiseptics and disinfectants, electrolytic, caloric and water balance agents, enzymes, antitussive, expectorants and mucolytic agents, gastric antisecretory agents, antiulcer agent, H₂ receptor antagonists, antidiarrhea agents, antiflatulent agents, cathartics and laxatives, gastrointestinal permeation enhancers, antihistamine, antiviral agents, antineoplastic agents, antifungal agents, cavity and infection preventing agents, antianemic agents, hemostatic agents, hemorrheologic agents, cardiovascular agents, hypotensive agents, anticoagulants, antilipemic agents, angiogenic and antiangiogenic agents, anti-inflammatory agents, vasodilators, bronchodilators, alkaloids, vitamins, peptides and proteins, vaccines, live or killed bacteria and viruses, powdered or pulverized parts of plants, trees, flowers, fruits, buds, seeds, leaves, barks, stem, roots, and animal tissues with medicinal and/or biological properties, agents or extracts derived from whole or parts of plants, trees, flowers, fruits, buds, seeds, leaves, barks, stem, roots, and animal tissues, growth promoting agents, soft and hard tissues, growth promoting agents, natural tissues such as bones or agents derived there from, bone growth promoting agents such as calcium phosphates, calcium sulfate and hydroxyapatites, genes, deoxyribonucleic acid (DNA), DNA fragments, ribonucleic acid (RNA), RNA fragments, and biological tissues. The BAS may also include other compounds such as those used in personal care products, such as cosmetics, flavors, fragrances, cleansing agents, and shampoos.

The invention is further described in the following non-limiting examples.

### EXAMPLE 1

Formulations 1 to 3 (as shown in Table 3) were prepared with different concentrations of CARBOPOL® 971P (Noveon, Inc., Cleveland, Ohio) using GELUCIRE® 50/13 (Gattefosse Corp., Westwood, N.J.) as the binder. An appropriate amount of Carbopol 971P was blended with Gelucire® 50/13, and lactose monohydrate (Spray dried lactose 315, Foremost Farms, Baraboo, Wis.) in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules were allowed to cool down to room temperature. An appropriate amount of propranolol HCl (BAS) was added and mixed with the free flowing granules in a V-shell blender and compressed into ⅜ inch Tablets on a HT-AP 18 SS-U/I rotary tablet press (Elizabeth Hata International, Inc., North Huntingdon, Pa.) to obtain 400 mg tablets.

TABLE 3
Composition of controlled release formulations prepared with an
intra-granular filler and extra-granular drug with varying concentrations
of Carbopol ® using Gelucire 50/13 as binder

| | | Formula- | Formula- | Formula- |
|---|---|---|---|---|
| | Ingredients | tion 1 | tion 2 | tion 3 |
| Granules | Carbopol ® 971P | 10% | 20% | 30% |
| | Gelucire ® 50/13 | 8% | 12% | 16% |
| | Lactose SD315 | 62% | 48% | 34% |
| Extra-Granular | Propranolol | 20% | 20% | 20% |
| Drug | HCl | | | |

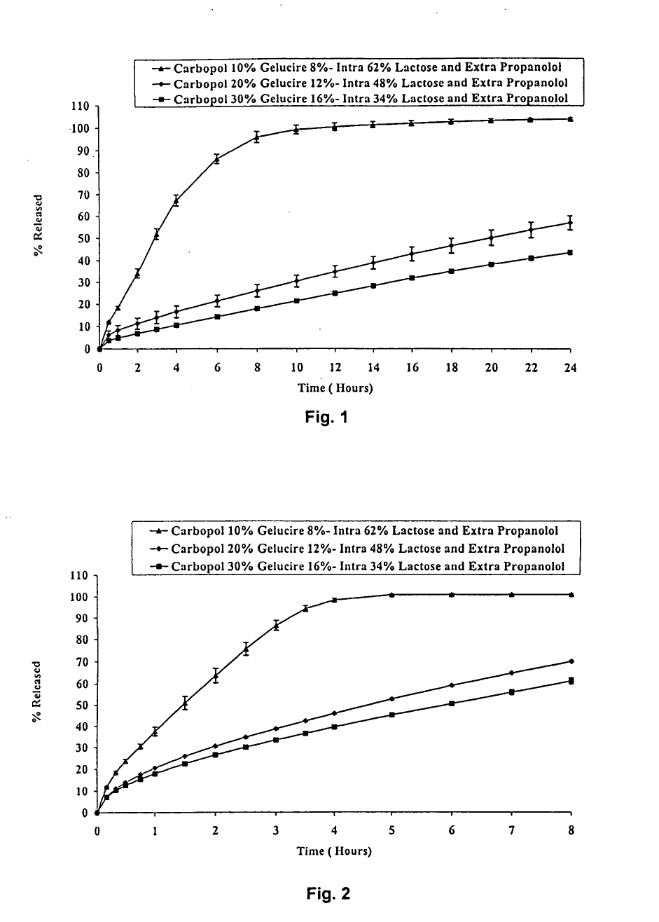

The test results of weight variation, thickness and hardness of the tablets are shown in Table 4. The effect of the polymer (carbomer) concentration on drug release characteristics in simulated intestinal fluid and in simulated gastric fluid is shown in FIGS. 1 and 2, respectively.

TABLE 4
Test results of weight variation, thickness and hardness of the tablets
compressed from granules obtained from Formulations 1-3

| Polymer | CF | Weight | RSD | Thickness | Hardness |
|---|---|---|---|---|---|
| Conc. w/w | (mTon) | (mg) | (%) | (mm) | (Kp) |
| 10% | 1.2 | 406.92 | 0.32 | 5.3368 | 6.38 |
| | 1.6 | 407.70 | 0.41 | 5.2944 | 7.14 |
| | 2.0 | 407.68 | 0.45 | 5.2392 | 8.80 |
| | 2.5 | 409.52 | 0.12 | 5.2304 | 9.82 |
| 20% | 0.8 | 403.44 | 0.52 | 5.4952 | 3.40 |
| | 1.0 | 406.44 | 0.95 | 5.4432 | 4.78 |
| | 2.0 | 405.60 | 1.06 | 5.3452 | 7.08 |
| | 2.5 | 401.88 | 0.71 | 5.268 | 8.60 |
| | 3.0 | 400.66 | 1.08 | 5.2264 | 8.74 |
| 30% | 1.6 | 400.30 | 0.13 | 5.4692 | 6.02 |
| | 2.0 | 408.74 | 0.52 | 5.5008 | 7.50 |
| | 2.5 | 402.82 | 0.16 | 5.3816 | 9.02 |

CF: compression force
RSD: relative standard deviation

### EXAMPLE 2

Formulations 4 to 6 were prepared with different concentrations of Carbopol® 971P using Sucrose Ester Fatty Acid (SEFA) behenate as the granulating binder (Table 5).

TABLE 5
Composition of the controlled release matrix tablets
prepared with varying Carbopol 971P concentrations using
intra-granular filler and extra-granular drug

| | | Formula- | Formula- | Formula- |
|---|---|---|---|---|
| | Ingredients | tion 4 | tion 5 | tion 6 |
| Granules | Carbopol ® 971P | 10% | 20% | 30% |
| | SEFA Behenate | 8% | 12% | 16% |
| | Lactose | 62% | 48% | 34% |
| Extra-Granular | Propranolol | 20% | 20% | 20% |
| Drug | HCl | | | |

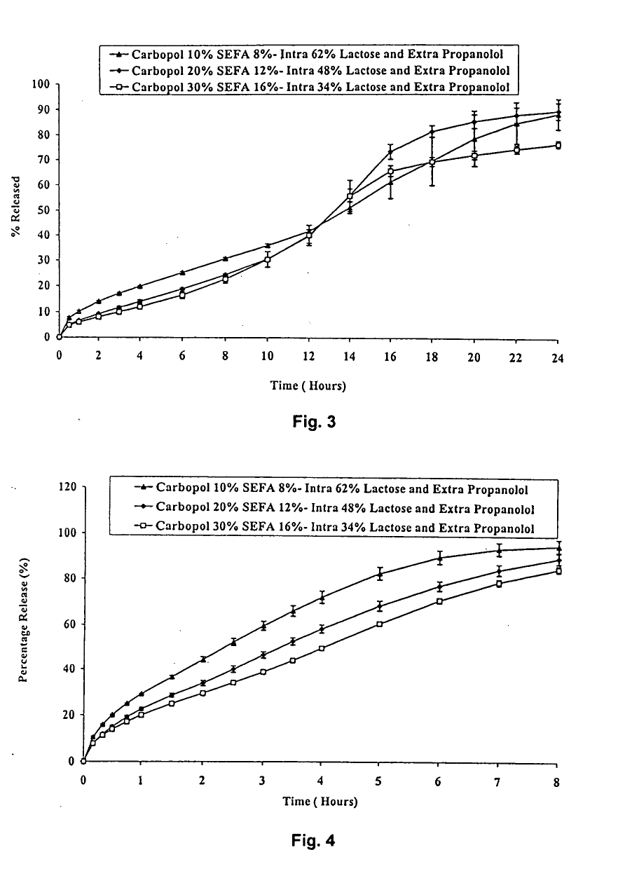

Carbopol® 971P was blended with SEFA behenate, and lactose monohydrate (Spray dried lactose 315, Foremost Farms, Baraboo, Wis.) in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules were allowed to cool down to room temperature. An appropriate amount of propranolol HCl (BAS) was added and mixed with the free flowing granules in a V-shell blender and compressed into ⅜ inch Tablets on a HT-AP 18 SS-U/I rotary tablet press (Elizabeth Hata International, Inc., North Huntingdon, Pa.) to obtain 400 mg tablets. The test results of weight variation, thickness and hardness of the tablets are shown in Table 6. The effect of the polymer concentration on drug release characteristics in simulated intestinal fluid and in simulated gastric fluid is shown in FIGS. 3 and 4 respectively.

TABLE 6
Test results of weight variation, thickness and hardness
of the tablets compressed from Formulations 4 to 6

| Polymer | CF | Weight | RSD | Thickness | Hardness |
|---|---|---|---|---|---|
| Conc. w/w | (mTon) | (mg) | (%) | (mm) | (Kp) |
| 10% | 0.4 | 402.28 | 0.47 | 5.6116 | 4.16 |
| | 0.6 | 408.56 | 0.27 | 5.3980 | 7.32 |
| | 0.8 | 406.00 | 0.49 | 5.2764 | 8.84 |
| 20% | 0.3 | 405.1 | 0.38 | 5.8144 | 3.46 |
| | 0.6 | 403.08 | 0.64 | 5.4420 | 7.42 |
| 30% | 0.6 | 413.40 | 0.71 | 5.6496 | 6.76 |
| | 1.0 | 416.40 | 0.59 | 5.5432 | 10.90 |
| | 2.0 | 416.66 | 0.56 | 5.4916 | 13.16 |

CF: compression force
RSD: relative standard deviation

### EXAMPLE 3

The formulations shown in Table 7 were prepared to study the effect of different types of binders on drug release characteristics.

TABLE 7
Composition of the controlled release formulations prepared with
different types of binders using intra-granular drug and filler

| | | Percent- | | Total |
|---|---|---|---|---|
| | | age in | Weight Per | Batch |
| Material | Ingredients | Tablets (%) | Tablet (mg) | Size (g) |
| Granules | Carbopol ® 971P | 15 | 60 | 150 |
| | Binder* | 10 | 40 | |
| | Propranolol HCL | 20 | 80 | |
| | Lactose SD315 | 54 | 216 | |
| Glidant | Cab-O-Sil | 0.5 | 2 | 3 |
| Lubricant | Magnesium | 0.5 | 2 | 3 |
| | Stearate | | | |
| Total | | 100 | 400 | 600 |
| Binder*: (1) Gelucire ® 50/13; (2) SEFA Behenate; (3) DYNASAN ® 116 Palmitic acid triglyceride); (4) Carnauba wax; (5) Sterotex ® NF (hydrogenated vegetable oil); (6) IMWITOR ® 900 (Glyceryl stearate); (7) PEG-20 Stearate; (8) Cithrol ® GMS 0400 (Glyceryl stearate); | | | | |
| # (9) Polawax ® A-31 (Fatty alcohol/sorbitan ester blend); (10) Syncrowax ® HR-C (Glyceryl Tribehenate); (11) Polawax ® NF (Fatty alcohol/polysorbate blend); (12) Crodamol ® SS (Cetyl esters wax, mixture of cetyl stearate, cetyl palmitate, and cetyl myristate); (13) Crodesta ® F-10 (sucrose distearate). | | | | |

An appropriate amount of Carbopol 971P according to Table 7 was blended with a hot melt binder, propranolol HCl and lactose monohydrate (Spray dried lactose 315, Foremost Farms, Baraboo, Wis.) in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules wee allowed to cool down to room temperature. An appropriate amount of lubricant (magnesium stearate) and glidant (Cab-O-Sil®) (Cabot Corporation, Tuscola, Ala., USA) was mixed with the free flowing granules and the resulting mixture was compressed into ⅜ inch 400 mg tablets on a HT-AP 18 SS-U/I rotary tablet press (Elizabeth Hata International, Inc., North Huntingdon, Pa.). The test results of weight variation, thickness and hardness of the tablets are shown in Table 8. The effect of different types of binders on drug release characteristics in simulated intestinal fluid is shown in FIG. 5.

TABLE 8
Test results of weight variation, thickness and hardness of the
tablets compressed from the formulations prepared with different
types of binders containing intra-granular drug and filler.

| | CF | Weight | RSD | Thickness | Hardness |
|---|---|---|---|---|---|
| Binder | (mTon) | (mg) | (%) | (mm) | (Kp) |
| Gelucire ® | 1.5 | 407.90 | 0.35 | 5.2984 | 12.18 |
| 50/13 | 2.6 | 407.26 | 0.22 | 5.2428 | 14.36 |
| | 3 | 407.08 | 0.18 | 5.2244 | 14.78 |
| | 3.2 | 406.48 | 0.46 | 5.234 | 14.52 |
| SEFA Behenate | 0.4 | 409.84 | 0.21 | 5.6715 | 5.8 |
| | 0.6 | 412.66 | 0.15 | 5.4876 | 8.88 |
| | 0.8 | 414.46 | 0.14 | 5.4056 | 11.24 |
| | 1 | 408.00 | 0.60 | 5.2556 | 13.82 |
| DYNASAN ® 116 | 0.2 | 400.84 | 0.19 | 5.8528 | 4.74 |
| | 0.4 | 401.76 | 0.17 | 5.5072 | 7.88 |
| | 0.6 | 404.32 | 0.24 | 5.3628 | 11.04 |
| | 0.8 | 408.94 | 0.32 | 5.298 | 12.46 |
| Carnauba wax | 0.4 | 413.82 | 0.31 | 5.844 | 4.92 |
| | 0.6 | 415.3 | 0.31 | 5.6032 | 8.66 |
| | 0.8 | 415.18 | 0.11 | 5.5052 | 11.1 |
| Sterotex ® NF | 0.3 | 399.6 | 0.08 | 5.6376 | 5.4 |
| | 0.5 | 399.72 | 0.26 | 5.3712 | 8.42 |
| | 0.8 | 403.56 | 0.18 | 5.2452 | 11.08 |
| IMWITOR ® 900 | 1.0 | 406.92 | 0.32 | 5.3368 | 6.38 |
| | 2.0 | 407.7 | 0.41 | 5.2944 | 7.14 |
| | 3.0 | 407.68 | 0.45 | 5.2392 | 8.8 |
| PEG-20 Stearate | 2.5 | 398.04 | 0.12 | 5.154 | 8.74 |
| | 3.3 | 405.86 | 0.95 | 5.1936 | 10.08 |
| Cithrol ® GMS | 0.2 | 400.56 | 0.38 | 5.8052 | 3.62 |
| 400 | 0.4 | 404.08 | 0.38 | 5.4655 | 6.96 |
| | 0.5 | 409.06 | 0.21 | 5.4324 | 7.86 |
| | 0.7 | 405.92 | 0.29 | 5.2708 | 9.9 |
| Polawax ® A-31 | 0.2 | 398.32 | 0.23 | 5.9624 | 3.36 |
| | 0.4 | 409.98 | 0.27 | 5.6884 | 7.38 |
| | 0.5 | 403.38 | 0.27 | 5.4816 | 9.22 |
| | 0.6 | 411.78 | 0.49 | 5.48 | 10.8 |
| Syncrowax ® | 0.4 | 410.22 | 0.44 | 5.6788 | 6.34 |
| HR-C | 0.5 | 395.10 | 0.93 | 5.3920 | 7.72 |
| | 0.6 | 420.70 | 0.17 | 5.5580 | 10.12 |
| | 1 | 415.28 | 0.26 | 5.3668 | 13.32 |
| Polawax ® NF | 0.2 | 405.08 | 0.49 | 6.1500 | 3.14 |
| | 0.4 | 410.26 | 0.30 | 5.6972 | 6.68 |
| | 0.5 | 412.36 | 0.50 | 5.6168 | 8.76 |
| | 0.6 | 414.86 | 0.24 | 5.5612 | 10.32 |
| Crodamol ® SS | 0.2 | 403.00 | 0.67 | 5.9910 | 3.85 |
| | 0.4 | 406.46 | 0.37 | 5.5692 | 7.34 |
| | 0.6 | 411.34 | 0.52 | 5.4440 | 9.88 |
| | 0.8 | 409.50 | 0.30 | 5.3240 | 11.68 |
| Crodesta ® F-10 | 0.5 | 401.48 | 0.22 | 5.4080 | 6.12 |
| | 0.7 | 403.48 | 0.65 | 5.3124 | 7.90 |
| | 0.9 | 394.48 | 1.23 | 5.1624 | 9.18 |

CF: compression force
RSD: relative standard deviation

### EXAMPLE 4

Table 9 shows the composition of the controlled release formulations containing extra-granular drug and filler and prepared with different types of binders.

TABLE 9
Composition of the controlled release matrix tablets
prepared with different types of binders, and containing
extra-granular drug and filler

| | | Percent- | | Total |
|---|---|---|---|---|
| | | age in | Weight Per | Batch |
| Material | Ingredients | Tablets (%) | Tablet (mg) | Size (g) |
| Granules | Carbopol ® 971P | 15 | 60 | 150 |
| | Binder* | 10 | 40 | |
| Extra- | Propranolol HCL | 20 | 80 | 120 |
| Granular | | | | |
| Drug | | | | |
| Extra- | Lactose SD 315 | 54 | 216 | 324 |
| Granular | | | | |
| Filler | | | | |
| Glidant | Cab-O-Sil | 0.5 | 2 | 3 |
| Lubricant | Magnesium | 0.5 | 2 | 3 |
| | Stearate | | | |
| Total | | 100 | 400 | 600 |

Binder* - Cithrol ® GMS 400; Polawax ® A-31; Syncrowax ® HR-C; Polawax ® NF; Crodamol ® SS; Crodesta ® F-10

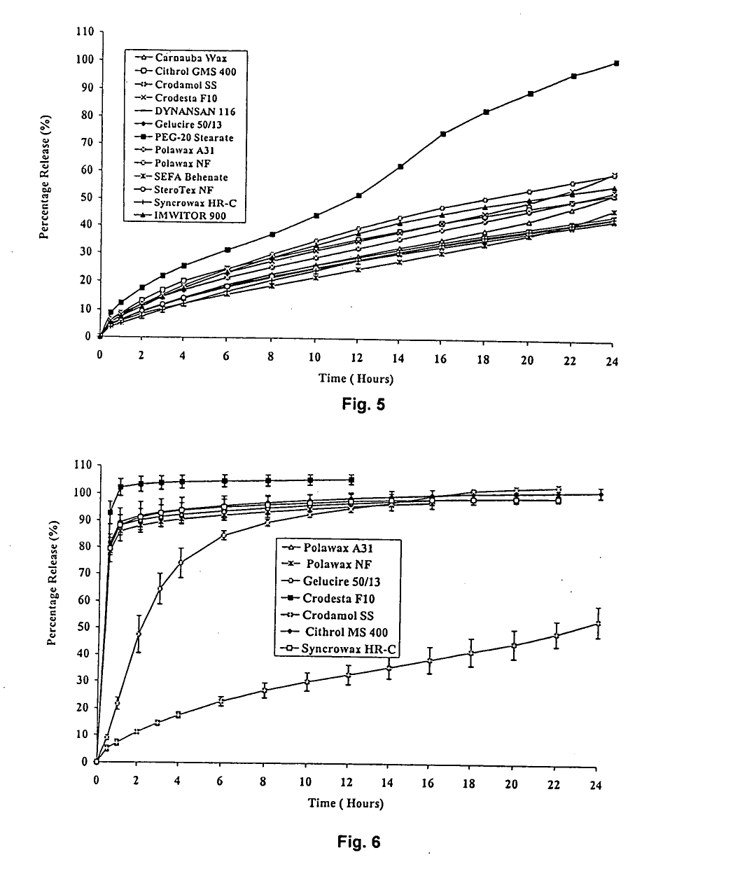

An appropriate amount of Carbopol® 971P was blended with a binder in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules were allowed to cool down to room temperature. The appropriate amount of extra-granular filler, propranolol HCl and Cab-O-Sil were blended with the obtained granules in a V-shell blend for 5 minutes. Magnesium stearate was added into the powder mixture and mixed for an additional 2 minutes. The obtained powder mixture was compressed into ⅜ inch tablets on a HT-AP 18 SS-U/I rotary tablet press (Elizabeth Hata International, Inc., North Huntingdon, Pa.) to obtain 400 mg tablets. The test results of weight variation, thickness, and hardness of the tablets are shown in Table 10. The effect of the type of binder on the drug release characteristics in simulated intestinal fluid is shown in FIG. 6.

TABLE 10
Test results of weight variation, thickness and hardness of the
tablets compressed from formulations prepared with different types
of binders and containing extra-granular drug and filler

| | CF | Weight | RSD | Thickness | Hardness |
|---|---|---|---|---|---|
| Binder | (mTon) | (mg) | (%) | (mm) | (kP) |
| Cithrol ® | 0.6 | 401.74 | 0.39 | 5.4944 | 5.00 |
| GMS 400 | 1.0 | 409.84 | 0.26 | 5.4204 | 8.08 |
| | 1.4 | 407.66 | 0.37 | 5.3184 | 9.44 |
| | 1.8 | 404.66 | 0.42 | 5.2588 | 10.10 |
| Polawax ® A-31 | 0.6 | 410.48 | 0.43 | 5.6384 | 5.82 |
| | 1 | 410.14 | 0.21 | 5.4692 | 8.72 |
| | 1.4 | 405.08 | 0.30 | 5.3320 | 11.02 |
| | 1.8 | 402.96 | 0.25 | 5.2628 | 12.42 |
| Syncrowax ® R-C | 0.6 | 406.12 | 0.43 | 5.5896 | 5.3 |
| | 1 | 410.94 | 0.46 | 5.486 | 8.06 |
| | 1.4 | 412.04 | 0.17 | 5.3932 | 11.48 |
| | 1.8 | 404.6 | 0.85 | 5.298 | 13.6 |
| Polawax ® NF | 0.6 | 408.94 | 0.32 | 5.6064 | 5.82 |
| | 1 | 410.22 | 0.58 | 5.4672 | 8.86 |
| | 1.4 | 408.48 | 0.35 | 5.3688 | 10.58 |
| | 1.8 | 402.74 | 0.21 | 5.2776 | 12.76 |
| Crodamol ® SS | 0.6 | 406.20 | 0.42 | 5.5332 | 6.32 |
| | 1.0 | 405.78 | 0.21 | 5.3876 | 9.52 |
| | 1.4 | 404.48 | 0.55 | 5.3028 | 11.98 |
| | 1.8 | 405.12 | 0.20 | 5.2764 | 12.22 |
| Crodesta ® F-10 | 0.6 | 402.16 | 0.22 | 5.4648 | 4.52 |
| | 1.0 | 410.44 | 0.35 | 5.4348 | 7.78 |
| | 1.4 | 410.26 | 1.19 | 5.3636 | 8.36 |

CF: compression force
RSD: relative standard deviation

### EXAMPLE 5

Table 11 shows the composition of the controlled release matrix tablets prepared with different binder concentrations and containing intra-granular filler and extra-granular drug.

TABLE 11
Composition of the controlled release matrix tablets
prepared with different binder concentrations and containing
intra-granular filler and extra-granular drug

| Material | Ingredients | | | |
|---|---|---|---|---|
| Granules | Carbopol ® 971P | 15% | 15% | 15% |
| | Gelucire ® 50/13 | 10% | 15% | 20% |
| | Lactose | 54% | 49% | 44% |
| Extra- | Propranolol HCL | 20% | 20% | 20% |
| Granular | | | | |
| Drug | | | | |
| Glidant | Cab-O-Sil | 0.5 | 0.5 | 0.5 |
| Lubricant | Magnesium | 0.5 | 0.5 | 0.5 |
| | Stearate | | | |
| Total | | 100 | 100 | 100 |

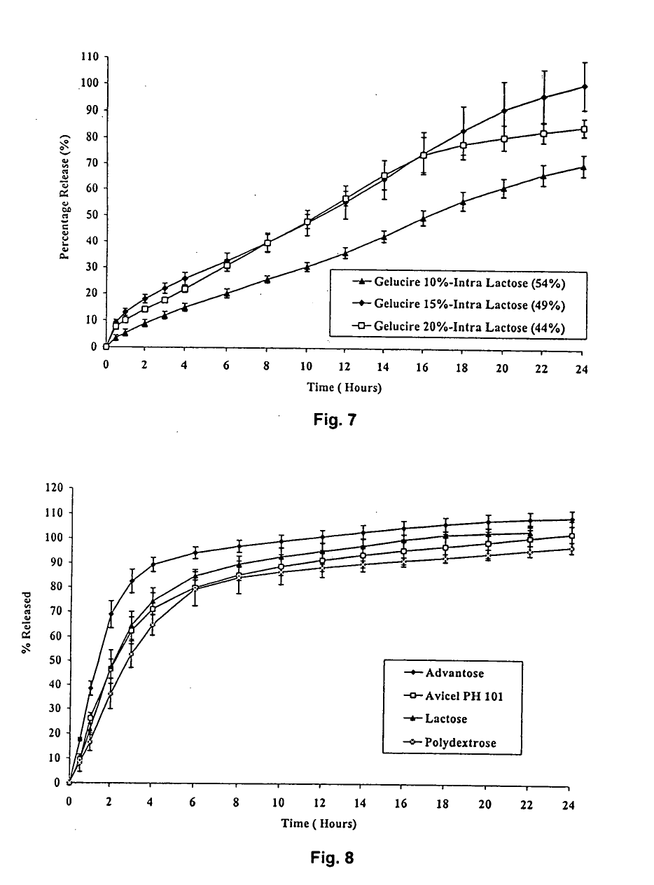

An appropriate amount of Carbopol 971P as shown in Table 11 was blended with Gelucire® 50/13 and lactose monohydrate (Spray dried lactose 315, Foremost Farms, Babraboo, Wis.) in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules were allowed to cool down to room temperature. The appropriate amount of extra-granular propranolol HCl and Cab-O-Sil were blended with the obtained granules in a V-shell blender for 5 minutes. Magnesium stearate was added into the powder mixture and mixed for an additional 2 minutes. The obtained powder mixture was compressed into ⅜ inch Tablets on a HT-AP 18 SS-U/I rotary tablet press (Elizabeth Hata International, Inc., North Huntingdon, Pa.) to obtain 400 mg tablets. The test results of weight variation, thickness, and hardness of the tablets are shown in Table 12. The effect of different types of binders on the drug release characteristics in simulated intestinal fluid is shown in FIG. 7.

Depending on the binder concentration, type of the binder, filler and drug, controlled drug release from the formulations prepared with extra-granular addition of the drug and filler is possible (FIGS. 6 and 7).

TABLE 12
Test results of weight variation, thickness and hardness of the
matrix tablets prepared with different binder concentrations and
containing intra-granular filler and extra-granular drug

| | CF | Weight | RSD | Thickness | Hardness |
|---|---|---|---|---|---|
| Gelucire ® 50/13 | (mTon) | (mg) | (%) | (mm) | (Kp) |
| 10% | 2.0 | 411.54 | 0.18 | 5.3276 | 7.80 |
| | 2.5 | 411.26 | 0.36 | 5.2924 | 9.82 |
| | 3.0 | 412.10 | 0.21 | 5.2916 | 9.26 |
| 15% | 1.8 | 403.54 | 0.21 | 5.3368 | 6.18 |
| | 2.5 | 407.88 | 0.48 | 5.3800 | 6.66 |
| | 3.0 | 409.22 | 0.50 | 5.3996 | 6.68 |
| 20% | 2.0 | 401.92 | 0.25 | 5.3220 | 6.42 |
| | 2.5 | 402.60 | 0.21 | 5.3268 | 6.50 |
| | 3.3 | 399.32 | 1.35 | 5.3272 | 6.56 |

CF: compression force
RSD: relative standard deviation

### EXAMPLE 6

Table 13 shows the composition of the controlled release formulations prepared with different types of extra-granular fillers and drug

TABLE 13
Composition of the controlled release matrix tablets containing
extra-granularly added drug and different types of fillers

| | | Quantity in | Weight Per | Total Batch |
|---|---|---|---|---|
| Material | Ingredients | Tablets (%) | Tablet (mg) | Size (g) |
| Granules | Carbopol ® 971P | 15 | 60 | 80.4 |
| | Gelucire ® 50/13 | 11.8 | 47.2 | |
| Extra- | Propranolol HCl | 20 | 80 | 60 |
| Granular | | | | |
| Drug | | | | |
| Extra- | Extra-Granular | 52.2 | 208.8 | 156.6 |
| Granular | Filler* | | | |
| Filler* | | | | |
| Glidant | Cab-O-Sil | 0.5 | 2 | 1.5 |
| Lubricant | Magnesium | 0.5 | 2 | 1.5 |
| | Stearate | | | |
| Total | | 100 | 400 | 300 |

Extra-Granular Filler*: Starch 1500 ®, Avicel ® PH 101, Advantose ® 300; Polydextrose; Lactose monohydrate, (Spray Dried Lactose 315)

An appropriate amount of Carbopol® 971P as shown in Table 13 was blended with Gelucire® 50/13 in a Robot-Coupe (Model 3 VG) high-shear mixer granulator for 2 minutes at 1000 rpm in the forward mode. After blending the powders, the high-shear mixer granulator was operated at 2000 rpm in the reverse mode until the end-point of the melt granulation process was reached. The formed granules were immediately removed from the granulator and passed through a 20-mesh screen. The screened granules were allowed to cool down to room temperature. The appropriate amounts of extra-granular filler, propranolol HCl and Cab-O-Sil were blended with the obtained granules in a V-shell blender for 5 minutes. Magnesium stearate was added into the powder mixture and mixed for an additional 2 minutes. The obtained powder mixture was compressed into ⅜ inch Tablets on a HT-AP 18 SS-U/I rotary tablet press Elizabeth Hata International, Inc., North Huntingdon, Pa.) to obtain 400 mg tablets. The test results of weight variation, thickness, and hardness of the tablets are shown in Table 14. The effect of different types of extra-granular filler on the drug release characteristics in simulated intestinal fluid is shown in FIG. 8.

TABLE 14
Test result of weight variation, thickness and hardness
of the tablets prepared with extra-granularly added
drug and different types of fillers

| | CF | Weight | RSD | Thickness | Hardness |
|---|---|---|---|---|---|
| Extra-granular | (mTon) | (mg) | (%) | (mm) | (Kp) |
| Avicel ® PH 101 | 0.4 | 390.00 | 0.92 | 6.1464 | 3.22 |
| | 0.8 | 402.96 | 0.32 | 5.6212 | 8.56 |
| | 1.2 | 397.86 | 0.48 | 5.4388 | 8.98 |
| Advantose ® 300 | 0.5 | 425.76 | 0.39 | 6.2244 | 7.82 |
| | 0.8 | 422.68 | 0.50 | 5.8712 | 11.28 |
| | 1.2 | 417.12 | 0.70 | 5.5912 | 16.88 |
| Lactose SD 315 | 1.6 | 394.58 | 0.65 | 5.2328 | 6.82 |
| | 2.0 | 401.28 | 0.55 | 5.2808 | 7.46 |
| | 3.0 | 402.76 | 0.43 | 5.3464 | 7.7 |
| Polydextrose | 2.0 | 404.56 | 0.18 | 5.4196 | 5.1 |
| | 2.5 | 414.68 | 0.53 | 5.5232 | 7.92 |
| | 3.0 | 412.64 | 0.31 | 5.4404 | 6.74 |
| Starch 1500 | 2.0 | 404.90 | 0.89 | 5.8112 | 2.76 |
| | 3.0 | 400.62 | 0.80 | 5.6932 | 3.38 |

CF: compression force
RSD: relative standard deviation

Further modifications, uses, and applications of the invention described herein will be apparent to those skilled in the art. It is intended that such modifications be encompassed in the following claims.

1. A method for making granules containing a carbomer comprising combining a carbomer with a hot-melt binder which is a solid or semi-solid at room temperature, subjecting the hot-melt binder to a temperature at which the binder melts or becomes tacky, wherein the combining of the carbomer and the hot-melt binder is either before the subjecting of the hot-melt binder to the temperature or is at a time when the binder is at a temperature at which it will melt or become tacky, and wherein the combining of the binder with the carbomer does not result in the lowering of more than 20° C. of the glass transition temperature of the carbomer, thereby obtaining an agglomerated powder, and permitting granules to form from the agglomerated powder.

2. The method of claim 1 wherein the combining of the binder with the carbomer does not result in the lowering of the glass transition temperature of the carbomer.

3. The method of claim 1 wherein the carbomer is selected from the group consisting of homopolymers of acrylic acid crosslinked with allyl sucrose or allyl pentaerythritol, polycarbophils of acrylic acid crosslinked with divinyl glycol, and copolymers of acrylic acid with minor levels of long chain alkyl acrylate crosslinked with allyl pentaerythritol.

4. The method of claim 1 wherein the hot-melt binder is a wax.

5. The method of claim 1 wherein the carbomer that is combined with the hot-melt binder is a plurality of carbomers.

6. The method of claim 1 wherein the hot-melt binder that is combined with the carbomer is a plurality of hot-melt binders.

7. The method of claim 6 wherein one or more of the hot-melt binders, if present as the sole hot-melt binder in the mixture, would lower the glass transition temperature of carbomer more than 20° C., but wherein the plurality of hot-melt binders in the mixture does not lower the glass transition temperature of carbomer more than 20° C.

8. The method of claim 1 wherein the hot-melt binder, when mixed by itself with a carbomer, would lower the glass transition temperature of carbomer more than 20° C., but wherein the hot-melt binder is combined with one or more additives to produce a combination that, when mixed with a carbomer, does not lower the glass transition temperature of the carbomer by more than 20° C.

9. The method of claim 1 wherein the mixture further contains a biologically active substance.

10. The method of claim 1 wherein the mixture further contains a controlled release agent, other than a carbomer.

11. The method of claim 10 wherein the controlled release agent is selected from the group consisting of a polymer, a gum, and a wax.

12. The method of claim 1 wherein the concentration of the hot-melt binder in the mixture is below that which will result in overwetting of the mixture.

13. The method of claim 1 wherein the concentration of the carbomer in the mixture is between 5% and 30% w/w.

14. The method of claim 1 wherein the mixture is substantially free of a biologically active substance.

15. A granule made by the method of claim 1.

16. The granule of claim 15 which further comprises a biologically active substance.

17. The granule of claim 16 wherein the biologically active substance is extragranular.

18. The granule of claim 16 wherein the biologically active substance is intragranular.

19. A method for producing a controlled release tablet or capsule containing a biologically active substance comprising obtaining a mixture containing a carbomer and a hot-melt binder which binder is a solid or semi-solid at room temperature and which binder, when combined with the carbomer, does not lower the glass transition temperature of the carbomer more than 20° C., subjecting the binder to a temperature at which the binder melts or becomes tacky wherein the subjecting of the hot-melt binder to the temperature is when the carbomer and the hot-melt binder are combined in a mixture or is before the carbomer and hot-melt binder are mixed and the carbomer and the hot-melt binder are mixed at a time when the binder is at a temperature at which it will melt or become tacky, thereby obtaining an agglomerated powder, permitting granules to form from the agglomerated powder, and adding a biologically active substance intragranularly or extragranularly to the formed granules.

20. The method of claim 19 wherein the combining of the binder and the carbomer does not lower the glass transition temperature of the carbomer.

21. A controlled release tablet or capsule made by the method of claim 19.

22. A capsule comprising a multiplicity of the granules of claim 15.

23. A tablet made by compressing a multiplicity of the granules of claim 15.
